(19) United States
(12) Patent Application Publication
(10) Pub. No.: US 2004/0259104 A1
(43) Pub. Date: Dec. 23, 2004

(54) Test

(76) Inventors: Steven Willem Jan Lamberts, Rotterdam (NL); Elisabeth Francisca Charlotte Van Rossum, Rotterdam (NL); Frans Jan Willem Koper, Rotterdam (NL)

Correspondence Address: CLARK & ELBING LLP, 101 FEDERAL STREET, BOSTON, MA, 02110, US

(21) Appl. No.: 10/661,217

(22) Filed: Sep. 12, 2003

(30) Foreign Application Priority Data
Oct. 22, 2002 (GB) 0224559.5

(51) Int. Cl. C12Q 1/68

(52) U.S. Cl. 435/6

(57) ABSTRACT

The invention provides a method of determining the risk of an individual developing a metabolic disorder, the method comprising:

(i) detecting in a sample from the individual the presence or absence of the ER22/23EK polymorphism in the glucocorticoid receptor gene; and

(ii) determining the likelihood of the individual developing a metabolic disorder, wherein the presence of the ER22/23EK polymorphism is indicative of a low risk of developing the metabolic disorder and the absence of the ER22/23EK polymorphism is indicative of a high risk of developing the metabolic disorder.

### FIELD OF THE INVENTION

The present invention relates to methods of predicting the future health of an individual and of determining the dosage of a glucocorticoid when used as a medicament.

### BACKGROUND TO THE INVENTION

Glucocorticoids (GC) exert their effects via the glucocorticoid receptor (GR) and play an important regulatory role in physiology. Several patients have been described with partial forms of GC resistance. They show a wide spectrum of clinical symptoms such as hypertension, hypokaleric alkalosis, fatigue and hyperandrogenism.

Within the normal population there is a considerable amount of variability in the feedback sensitivity of the hypothalamo-pituitary-adrenal (HPA)-axis. The molecular mechanisms underlying this variation in GC-sensitivity are still largely unknown.

In the symptomatic patients with familial forms of glucocorticoid resistance, missense mutations in the ligand binding domain of the glucocorticoid receptor gene causing decreased ligand binding have been described, as well as a deletion of four base pairs at the boundary of exon 6 and intron 6 causing loss of a splice site and a 50% reduction in the number of receptors per cell.

Within the normal population, several polymorphisms in the GR gene have been reported. One of these polymorphisms consists of a point mutation in codon 363 in exon 2 of the GR gene and results in an asparagine to serine amino acid change which is associated with an increased sensitivity to GCs in response to dexamethasone (DEX). Another polymorphism (ER22/23EK) consists of two linked point mutations separated by one base pair in codons 22 and 23 in exon 2 of the GR gene. The first mutation is silent, changing codon 22 from GAG to GAA, both coding for glutamic acid (E). The second mutation changes codon 23 from AGG to AAG, resulting in an amino acid change from arginine (R) to lysine (K) (Koper et al. Hum. Genet. 1997; 99(5):663-8). These mutations have been shown not to alter the activity of GR in “in vitro” experiments (de Lange et al, Mol. Endocinol 1997; 11(8): 1156-64). The clinical relevance of this polymorphism has not been studied but carriers show a variety of phenotypes ranging from asymptomatic to glucocorticoid resistant (Huizenga et al., J. Clin. Endocinol. Metab. 2000; 85(5): 2076-81).

### SUMMARY OF THE INVENTION

The present inventors have investigated, within the context of an ongoing population based cohort study of diseases in the elderly (The Rotterdam Study), whether there are any differences in vivo between ER22/23 EK-carriers and non-carriers. They have found, unexpectedly, that there were a significantly higher percentage of ER22/23EK-carriers in the highest age group (age 67-82 years) than in the youngest age group (age 53-67 years) and thus that this GR polymorphism is associated with an increased life expectancy. They also found that after administration of dexamethasone (DEX) the ER22/23EK-carriers had higher serum cortisol concentrations than non-carriers and that ER22/23 EK-carriers had lower insulin levels than non-carriers both before and after DEX. In addition, they found that ER22/23EK-carriers tended to have lower fasting glucose concentrations and that cholesterol concentrations were significantly lower in the ER22/23EK-carriers.

Accordingly, the present invention provides:

a method of determining the risk of an individual developing a metabolic disorder, the method comprising:

(i) detecting in a sample from the individual the presence or absence of the ER22/23EK polymorphism in the glucocorticoid receptor gene; and

(ii) determining the likelihood of the individual developing a metabolic disorder, wherein the presence of the ER22/23EK polymorphism is indicative of a low risk of developing the metabolic disorder and the absence of the ER22/23EK polymorphism is indicative of a high risk of developing the metabolic disorder;

a method of predicting the longevity of an individual, the method comprising:

(i) detecting in a sample from the individual the presence or absence of the ER22/23EK polymorphism in the glucocorticoid receptor gene; and

(ii) determining the life expectancy of the individual, wherein the presence of the ER22/23EK polymorphism is indicative of a long life expectancy;

a method of determining the dose of glucocorticoid for administration to an individual in need thereof, the method comprising:

(i) detecting in a sample from the individual the presence or absence of the ER22/23EK polymorphism in the glucocorticoid receptor gene; and

(ii) determining whether the dose of glucocorticoid for administration to the individual should be altered compared to the standard dosage, wherein the presence of the ER22/23EK polymorphism indicates that the dosage should be increased;

a method of determining whether a treatment regimen is suitable for an individual having a metabolic disorder, the method comprising:

(i) detecting in a sample from the individual the presence or absence of the ER22/23EK polymorphism in the glucocorticoid receptor gene; and

(ii) determining whether the treatment is suitable for the individual, wherein the suitability of the treatment depends on the presence or absence of the ER22/23EK polymorphism;

a method for diagnosing and treating an individual susceptible to a metabolic disorder, the method comprising:

(i) detecting in a sample from the individual the presence or absence of the ER22/23EK polymorphism in the glucocorticoid receptor gene; and

(ii) administering to an individual having the ER22/23KK polymorphism a therapeutically effective amount of an agent which prevents or treats the metabolic disorder;

a method for increasing the life expectancy of an individual, the method comprising;

(i) detecting in a sample from the individual the presence or absence of the ER22/23EK polymorphism in the glucocorticoid receptor gene; and

(ii) introducing into the individual an allele of the glucocorticoid receptor gene or a glucocortidicoid receptor, wherein said gene or polypeptide does not have said polymorphism;

a method for identifying an agent comprising:

(i) contacting a glucocorticoid receptor polypeptide having the sequence shown in SEQ ID NO: 1 or a fragment thereof which includes the ER22/23EK polymorphism with a test agent;

(ii) monitoring binding of the test agent to the polypeptide; and

(iii) determining whether said test agent may increase life expectancy or be suitable for treating a metabolic disease, wherein for increasing life expectancy or treating a metabolic disease agent is one that binds to the polypeptide.

Brief Description of the Sequences

SEQ ID NO: 1 is the nucleotide sequence of the glucocorticoid receptor cDNA having the ER22/23EK polymorphism. SEQ ID NO: 2 is the amino acid sequence of the glucocorticoid receptor having the ER22/23EK polymorphism.

SEQ ID NO: 3 is the nucleotide sequence of the non-variant allele of the glucocorticoid receptor, i.e. the cDNA sequence not having the ER22/23EK polymorphism. SEQ ID NO: 4 is the amino acid sequence of the glucocorticoid receptor not having the ER22/23EK polymorphism.

### BRIEF DESCRIPTION OF THE FIGURES

FIG. 1 is a schematic representation of the structure of the human glucocorticoid receptor gene, mRNA and protein showing its functional domains. The position of the arginine to lysine change at codon 23 as a result of the G to A point mutation and the silent point mutation of a G to A at codon 22 are indicated. N-TERM=NH₂-terminal domain; DBD=DNA binding domain and HBD=hormone binding domain.

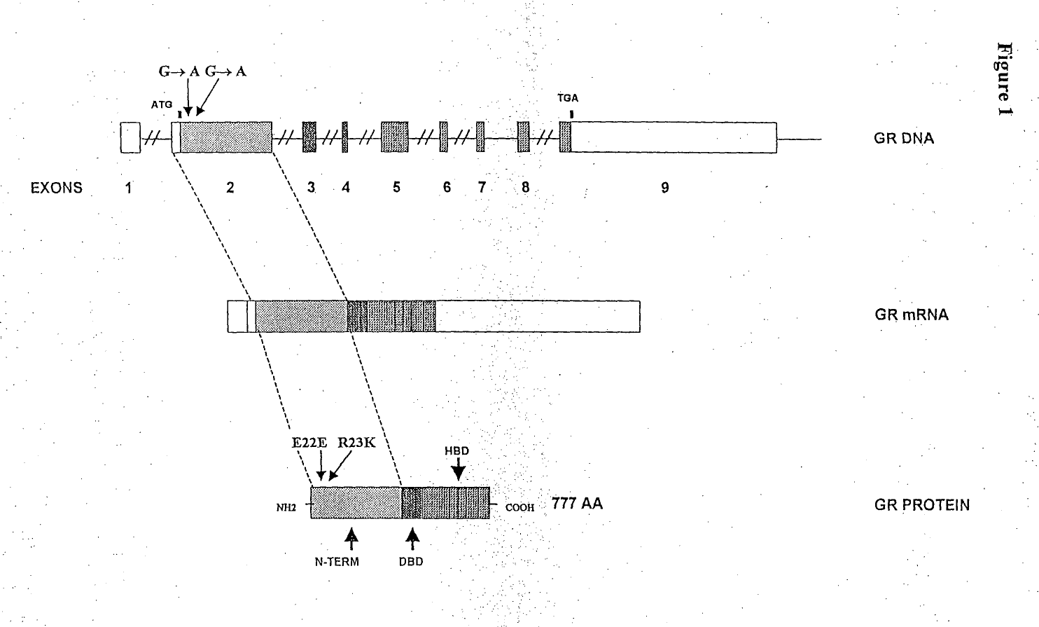

### DETAILED DESCRIPTION OF THE INVENTION

In the first aspect, the present invention provides a method for determining the risk of an individual developing a metabolic disorder, the method comprising:

(i) detecting in a sample from the individual the presence or absence of the ER22/23EK polymorphism in the glucocorticoid receptor gene; and

(ii) determining the likelihood of the individual developing a metabolic disorder, wherein the presence of the ER22/23EK polymorphism is indicative of a low risk of developing the metabolic disorder and the absence of the ER22/23EK polymorphism is indicative of a high risk of developing the metabolic disorder.

The method can be used to assess whether an individual may develop a metabolic disorder in the future. Therefore, the individual may be asymptomatic, ie. may not exhibit any symptoms associated with the metabolic disease, for example, the individual may not show any symptom of glucocorticoid resistance.

The metabolic disease is selected from cardiovascular disease, diabetes mellitus, glucose intolerance/insulin resistance, dyslipidemia (hypercholesterolemia in particular) and (metabolic) Syndrome X.

The metabolic disorder is preferably selected from cardiovascular disease, glucose-intolerance and/or diabetes mellitus.

The glucocorticoid receptor gene and the ER22/23EK polymorphism have been described previously and are shown in SEQ ID NO: 1 (polynucleotide) and SEQ ID NO: 2 (polypeptide).

An individual having the ER22/23EK polymorphism has an adenosine (A) residue at the second position in codon 23 and generally also an adenosine (A) residue at the third position in codon 22 of the glucocorticoid receptor gene (at positions 198 and 200 in SEQ ID NO. 1). Thus an individual having the ER22/23EK polymorphism will have a lysine (K) residue at position 23 of the glucocorticoid receptor amino acid sequence.

The risk of an individual developing a metabolic disease is the likelihood of the individual developing the metabolic disease in the future. The likelihood of developing a metabolic disease is greater in an individual not having a ER22/23EK polymorphism in the glucocorticoid receptor gene than in an individual having this polymorphism. Thus, detection of the presence of the ER22/23EK polymorphism in a sample from an individual indicates that the individual has a low chance of developing the metabolic disorder and detection of the absence of the ER22/23EK polymorphism constitutes a high risk.

An individual having the ER22/23EK polymorphism has a low risk of developing a metabolic disorder compared to an individual not having the ER22/23EK polymorphism. An individual not having the ER22/23EK polymorphism has a high risk of developing a metabolic disorder compared to an individual having the ER22/23EK polymorphism.

An individual with a low risk of developing a metabolic disorder typically has a 10 to 30%, preferably a 5 to 20% chance and more preferably a 0 to 15% chance of developing the disorder. An individual not having the ER2)/23EK polymorphism may be considered as being susceptible to a metabolic disorder.

Where it is determined that an individual does not have the ER22/23EK polymorphism, i.e. that the individual has a high risk of developing a metabolic disorder, further tests may be carried out on a sample from the individual and/or the individual may be prescribed a preventative treatment.

For example, the individual's lipid spectrum in the blood (especially total and LDL cholesterol), fasting glucose levels or blood pressure may be tested. The determination of other cardiovascular risk factors, such as smoking, increased weight and positive family history for cardiovascular disease at a relatively young age may be carried out to fully assess the individual's risk profile.

In a second aspect, the present invention provides a method of predicting the life expectancy of an individual by detecting in a sample from the individual the presence of the ER22/23EK polymorphism in the glucocorticoid receptor gene, wherein the presence of the ER22/23EK polymorphism is indicative of a long life expectancy.

Also provided by the invention, is a method for increasing the life expectancy of an individual, the method comprising detecting in a sample from the individual the presence or absence of the ER22/23EK polymorphism in the glucocorticoid receptor gene and introducing into the individual an allele of the glucocorticoid receptor gene or a glucocorticoid receptor polypeptide, which gene or polypeptide does not have the ER22/23EK polymorphism. The gene not having the ER22/23EK polymorphism is typically a polynucleotide which encodes a glucocorticoid receptor having an arginine residue at position 22. A glucocorticoid receptor polypeptide not having the ER22/23EK polymorphism typically has an arginine residue at position 22.

The life expectancy of an individual having the ER22/23EK polymorphism is typically greater than that of an individual not having the ER22/23EK polymorphism. Generally the presence of the ER22/23EK polymorphism in a sample from an individual indicates that the individual is expected to survive to the age of at least 67, 70 or 75 preferably 80 or 85 years of age.

The individual may be of any age, for example, from 15 to 25, 26 to 35, 36 to 45, 46 to 55, 56 to 65 or 66 to 75. Preferably the individual does not show any symptoms of a metabolic disorder.

In a further aspect, the present invention provides a method of determining the dose of glucocorticoid for administration to an individual in need thereof, the method comprising:

(i) detecting in a sample from the individual the presence or absence of the ER22/23EK polymorphism in the glucocorticoid receptor gene; and

(ii) determining whether the dose of glucocorticoid for administration to the individual should be altered compared to the standard dosage, wherein the presence of the ER22/23EK polymorphism indicates that the dosage should be increased.

The method may also comprise the further step of administering the glucocorticoid in the required dose, i.e. in a therapeutically effective amount, to the individual.

If the ER22/23EK polymorphism is present then the dose of glucocorticoid required will be raised compared to the normal dose. The normal dose for a particular route of administration is known to a skilled practitioner. A skilled practitioner will also be readily able to determine by how much the dose of glucocorticoid should be increased in an individual having the ER22/23EK polymorphism.

An individual in need of glucocorticoid administration is typically suffering from a disease which is generally treated by the administration of a glucocorticoid. Such diseases include: Allergic diseases such as asthma, atopic dermatitis and anaphylactic shock, autoimmune diseases such as systemic lupus erythematosus, rheumatoid arthritis, systemic vasculitis, polymyalgia rheumatica, temporal arteritis, Graves' ophthalmopathy, autoimmune hemolysis and myasthenia gravis, inflammatory disorders such as Crohn's disease and ulcerative colitis, neoplastic diseases such as lymphomas, graft rejection for example following kidney, heart, lung liver or other tissue transplantation, sarcoidosis, vitamin D intoxication, thyroid storm, septic shock, cerebral edema, altitude sickness, chronic bronchitis and emphysema.

Glucocorticoids that are typically administered to treat such diseases include: Hydrocortisone, Prednisone, Prednisolone, Methylprednisolone, Triamcinolone, Dexamethasone, Budesonide, Betamethasone and Beclomethasone.

In a further aspect, the present invention provides a method of determining whether a treatment regime is suitable for an individual having a metabolic disorder, the method comprising detecting in a sample from the individual the presence or absence of the ER22/23EK polymorphism in the glucocorticoid receptor gene; and determining whether the treatment will be suitable for the individual, wherein the suitability of the treatment depends on the presence or absence of the ER22/23EK polymorphism.

The individual having a metabolic disorder typically exhibits one or more symptom associated with a metabolic disorder. Symptoms of cardiovascular disease which may be exhibited include: nycturia, ankle edema, orthopnoea/dyspnoea, intermittent claudicatio, neurological symptoms as a result of stroke, nausea, sweating, unconsciousness and chest pain (and referred pain in left shoulder, jaw, or in between shoulders). Symptoms of diabetes mellitus include: polydipsia, polyuria, weight loss, polyphagia, abdominal pain, nausea and vomiting, drowsiness, Kussmaul respirations, dehydration and obesity. In cases of insulin resistance and dyslipedemia with very high circulating cholesterol levels xanthelasmata can be observed. Additional symptoms of Syndrome X include obesity, hyperlipidemia and hypertension.

The invention also provides a method for diagnosing and treating an individual susceptible to a metabolic disorder, the method comprising:

(i) detecting in a sample from the individual the presence or absence of the ER22/23EK polymorphism in the glucocorticoid receptor gene;

(ii) administering to an individual having the ER22/23EK polymorphism, a therapeutically effective amount of an agent which prevents or treats the metabolic disorder. The agent may be a glucocorticoid receptor gene polynucleotide or polypeptide which does not have the ER22/23EK polymorphism. The agent may be a glucocorticoid. Other suitable agents may be identified by a screening method of the invention.

A sample used in any one of the methods of the invention typically comprises a bodily fluid of the individual and may be obtained by any suitable method, for example by using a swab, such as a mouth swab. The sample may be a blood, urine, saliva, cheek cell or hair root sample. The sample is generally processed before the method is carried out, for example DNA may be extracted from the sample and used in the method of the invention. The polynucleotide or protein in the sample may be cleaved either physically or chemically (e.g. using a suitable enzyme).

The individual is typically a human. The individual may be male or female.

Detection of Polymorphisms

The presence or absence of the polymorphism may be detected using any method that allows the sequence of the glucocorticoid receptor gene at position 3 in codon 22 and/or position 2 in codon 23, or the sequence of the glucocorticoid receptor polypeptide at codon 23 or at codons 22 and 23 to be determined. The detection method may be chosen to determine the presence of the ER22/23EK polymorphism (the variant sequence), the presence of the non-variant sequence or both the variant and non-variant sequence.

The non-variant sequence is a glucocorticoid receptor coding polynucleotide sequence having a G at position 3 of codon 22 and a G at position 2 of codon 23, or a glucocorticoid receptor polypeptide sequence having an arginine residue at position 23. The variant sequence is a glucocorticoid receptor coding polynucleotide having an A at position 2 of codon 23 and/or at position 3 of codon 22, or a glucocorticoid receptor polypeptide having a lysine residue at position 23.

The polymorphism may be detected in the polynucleotide encoding the glucocorticoid receptor or in the glucocorticoid receptor polypeptide. The ER22/23EK polymorphism generally consists of two point mutations in the glucocorticoid receptor polynucleotide. The sequence at either one or both of the positions of the point mutations may be determined.

The presence or absence of the ER22/23EK polymorphism is typically detected by directly determining the presence or absence an allele of the polymorphic sequence in a polynucleotide or protein of the individual.

Thus, a method of the invention may comprise detection of a glucocorticoid receptor polynucleotide sequence having an adenosine (A) residue at the third position in codon 22 and/or an adenosine (A) residue at the second position in codon 23 (i.e. an adenosine residue at position 98 and/or position 100 of SEQ ID NO: 1). A glucocorticoid receptor polynucleotide sequence having a guanosine (G) residue at the third position in codon 22 and/or a guanosine (G) residue at the second position in codon 23 (i.e. a guanosine residue at position 98 and/or position 100 of SEQ ID NO: 1) may also be detected. Both alleles of the polymorphic sequence may be detected in a method of the invention, for example using a quantitative detection method or in a method using differently labelled probes, to determine whether the individual is heterozygous, homozygous for the ER22/23EK polymorphism or homozygous for the non-variant allele.

Where the glucocorticoid receptor protein sequence is used to detect the presence or absence of the ER22/23EK polymorphism, the glucocorticoid receptor sequence, or a fragment thereof, with a lysine residue at position 23 may be detected. Alternatively, the sequence detected may have an arginine residue at position 23. A method may involve detecting both a sequence comprising a lysine residue at position 23 and a sequence comprising an arginine residue at position 23.

A polynucleotide for detection is typically genomic DNA or mRNA, or a polynucleotide derived from these polynucleotides, such as in a library made using polynucleotide from the individual (e.g. a cDNA library). The processing of the polynucleotide or protein before the carrying out of the method is discussed further below.

Generally, the presence of the polymorphism is determined in a method that comprises contacting a polynucleotide or protein of the individual with a specific binding agent for the polymorphism and determining whether the agent binds to a polymorphism in the polynucleotide or protein, the binding of the agent to the polymorphism indicating that the ER22/23EK polymorphism is present in the sample.

Generally the agent will also bind to flanking nucleotides and amino acids on one or both sides of the polymorphism, for example at least 2, 5, 10, 15 or more flanking nucleotides or amino acids in total or on each side. Generally in the method, determination of the binding of the agent to the polymorphism can be done by determining the binding of the agent to the polynucleotide or protein. However in one embodiment the agent is able to bind the corresponding wild-type sequence by binding the nucleotides or amino acids which flank the polymorphism position, although the manner of binding will be different to the binding of a polynucleotide or protein containing the polymorphism, and this difference will generally be detectable in the method (for example this may occur in sequence specific PCR as discussed below).

In the case where the presence of the polymorphism is being determined in a polynucleotide it may be detected in the double stranded form, but is typically detected in the single stranded form.

The agent may be a polynucleotide (single or double stranded) typically with a length of at least 10 nucleotides, for example at least 15, 20, 30 or more polynucleotides. The agent may be a molecule which is structurally related to polynucleotides that comprises units (such as purines or pyrimidines) able to participate in Watson-Crick base pairing. The agent may be a polypeptide, typically with a length of at least 10 amino acids, such as at least 20, 30, 50, 100 or more amino acids. The agent may be an antibody (including a fragment of such an antibody which is capable of binding the polymorphism).

A polynucleotide agent which is used in the method will generally bind to the polymorphism, and flanking sequence, of the polynucleotide of the individual in a sequence specific manner (e.g. hybridise in accordance with Watson-Crick base pairing) and thus typically has a sequence which is fully or partially complementary to the sequence of the polymorphism and flanking region. The partially complementary sequence is homologous to the fully complementary sequence.

In one embodiment of the method the agent is as a probe. This may be labelled or may be capable of being labelled indirectly. The detection of the label may be used to detect the presence of the probe on (and hence bound to) the polynucleotide or protein of the individual. The binding of the probe to the polynucleotide or protein may be used to immobilise either the probe or the polynucleotide or protein (and thus to separate it from a composition or solution).

In one embodiment the polynucleotide or protein of the individual is immobilised on a solid support and then contacted with the probe. The presence of the probe immobilised to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism the method may be based on an ELISA system.

The method may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the presence of single mismatch within one of the probes may disrupt binding and ligation. Thus ligated probes will only occur with a polynucleotide that contains the polymorphism, and therefore the detection of the ligated product may be used to determine the presence of the polymorphism.

In one embodiment the probe is used in a heteroduplex analysis based system to detect polynucleotide polymorphisms. In such a system when the probe is bound to polynucleotide sequence containing the polymorphism it forms a heteroduplex at the site where the polymorphism occurs (i.e. it does not form a double strand structure). Such a heteroduplex structure can be detected by the use of an enzyme which is single or double strand specific. Typically the probe is an RNA probe and the enzyme used is RNAse H which cleaves the heteroduplex region, thus allowing the polymorphism to be detected by means of the detection of the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment the polynucleotide agent is able to act as a primer for a PCR reaction only if it binds a polynucleotide containing the ER22/23EK polymorphism (i.e. a sequence-specific or allele-specific PCR system). The primer may bind to the coding sequence or the complement of the coding sequence. The primer may thus be a fragment of the sequence shown in SEQ ID NO: 1 or may be complementary to a fragment of the sequence shown in SEQ ID NO: 1. Thus a PCR product will only be produced if the polymorphism is present in the polynucleotide of the individual. Thus the presence of the polymorphism may be determined by the detection of the PCR product. Preferably the region of the primer which is complementary to the polymorphism is at or near the 3′ end of the primer. In one embodiment of this system the polynucleotide agent will bind to the wild-type sequence but will not act as a primer for a PCR reaction.

Alternatively, the agent may be able to act as a primer for a PCR reaction only if it binds to a sequence not containing the ER22/23EK polymorphism, i.e. to a glucocorticoid receptor polynucleotide sequence comprising guanosine residues at position three of codon 22 and/or position two of codon 23.

The method may be an RFLP based system. This can be used if the presence of the polymorphism in the polynucleotide creates or destroys a restriction site which is recognised by a restriction enzyme. Thus treatment of a polynucleotide with such a polymorphism will lead to different products being produced compared to the corresponding wild-type sequence. Thus the detection of the presence of particular restriction digest products can be used to determine the presence of the polymorphism.

The presence of the polymorphism may be determined based on the change which the presence of the polymorphism makes to the mobility of the polynucleotide or protein during gel electrophoresis. In the case of a polynucleotide, single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DDGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the Taqman™ PCR detection system. Generally this assay uses an allele specific primer comprising the sequence around, and including, the polymorphism. The specific primer is labelled with a fluorescent dye at its 5′ end, a quenching agent at its 3′ end and a 3′ phosphate group preventing the addition of nucleotides to it. Normally the fluorescence of the dye is quenched by the quenching agent present in the same primer. The allele specific primer is used in conjunction with a second primer capable of hybridising to either allele 5′ of the polymorphism.

In the assay, when the allele comprising the polymorphism is present, Taq DNA polymerase adds nucleotides to the non-specific primer until it reaches the specific primer. It then releases nucleotides, the fluorescent dye and quenching agent from the specific primer through its endonuclease activity. The fluorescent dye is therefore no longer in proximity to the quenching agent and fluoresces. In the presence of the allele which does not comprise the polymorphism the mismatch between the specific primer and template inhibits the endonuclease activity of Taq and the fluorescent dye is not released from the quenching agent. Therefore, by measuring the fluorescence emitted the presence or absence of the polymorphism can be determined.

In another method of detecting the polymorphism, a polynucleotide comprising the polymorphic region is sequenced across the region which contains the polymorphism to determine the presence of the polymorphism.

Diagnostic Kit

The invention also provides a diagnostic kit that comprises an agent, probe, primer or antibody (including an antibody fragment) capable of detecting the ER22/23EK polymorphism in the glucocorticoid receptor gene or protein and instructions for using the agent, probe, primer or antibody in a method of the invention. The kit may additionally comprise one or more other reagents or instruments which enable any of the embodiments of the method mentioned above to be carried out.

Such reagents or instruments include one or more of the following: a means to detect the binding of the agent to the polymorphism, a detectable label (such as a fluorescent label), an enzyme able to act on a polynucleotide (typically a polymerase, restriction enzyme, ligase, RNAse H or an enzyme which can attach a label to a polynucleotide), suitable buffer(s) (aqueous solutions) for enzyme reagents, PCR primers which bind to regions flanking the polymorphism (e.g. the primers discussed herein), a positive and/or negative control, a gel electrophoresis apparatus, a means to isolate DNA from sample, a means to obtain a sample from the individual (such as swab or an instrument comprising a needle) or a support comprising wells on which detection reactions can be done.

Screening Methods

In a still further aspect, the present invention provides a method for identifying an agent for increasing life expectancy or treating a metabolic disease, which method comprises:

(i) contacting a glucocorticoid receptor polypeptide having the sequence shown in SEQ ID NO. 2 or a fragment thereof which includes the ER22/23EK polymorphism with a test agent and

(ii) monitoring binding of the test agent to the polypeptide; and

(iii) determining whether said test agent may increase life expectancy or be suitable for treating a metabolic disease, wherein a suitable agent is one that binds to the polypeptide.

A fragment for use in a method of the invention is typically at least 5 amino acids long, such as at least 10, 15, 20, 50 or 100 amino acids long.

The invention also provides the use of a non-human animal which is transgenic for a polynucleotide having the sequence shown in SEQ ID NO. 1 in screening for agents for use in the treatment of a metabolic disorder or for increasing life expectancy.

The transgenic non-human animal is generally a mammal. The transgenic non-human animal is typically of a species commonly used in biomedical research and is preferably a laboratory strain. Suitable animals include non-human primates and rodents. It is preferred that an animal for use in a screening method is a rodent, particularly a mouse, rat, guinea pig, ferret, gerbil or hamster. Most preferably the animal is a mouse.

A typical method for identifying an agent for use in the treatment of a metabolic disorder of increasing life expectancy consists essentially of:

(i) administering a test agent to a non-human animal which is transgenic for a polynucleotide having the sequence shown in SEQ ID NO. 1; and

(ii) monitoring a metabolic process.

The method may comprise the further step of sacrificing the animal.

Metabolic processes that may be monitored include pre- and post-DEX cortisol concentration, pre- and post-DEX insulin and/or glucose levels and total or LDL-cholesterol levels.

Suitable candidate agents which may be tested include antibody products (for example, monoclonal and polyclonal antibodies, single chain antibodies, chimeric antibodies and CDR grafted antibodies). Furthermore, combinatorial libraries, defined chemical entities, peptide and peptide mimetics, oligonucleotides and natural product libraries, such as display libraries may also be tested. The candidate agents may be used in an initial screen of, for example, ten substances per administration, and the agents of batches which show an effect on metabolite levels.

The term ‘agent’ is intended to include a single substance and a combination of two, three or more substances. For example, the term agent may refer to a single peptide, a mixture of two or more peptides or a mixture of a peptide and a defined chemical entity.

A transgenic non-human animal of the invention may be dosed with the test agent prophylactically or therapeutically on one or more occasions. Typically test agents may be administered bi-weekly, weekly, twice weekly, daily or two, three or more times a day, for example, at hourly or at two, three or four hourly intervals.

The test agents may be formulated with standard carriers and/or excipients as is routine in the pharmaceutical art, and as fully described in Remington's Pharmaceutical Sciences, Mack Publishing Company, Eastern Pennsylvania 17^{th} Ed. 1985.

A test agent may be administered by enteral or parenteral routes such as via oral, buccal, anal, intravenous, intra-arterial, intramuscular, intraperitoneal, subcutaneous or other appropriate administration routes. A test agent may be present in the food or drinking water or may be administered using an osmotic minipump.

Test agents may be administered at any appropriate dosage. A typical dose may be from 0.1 to 50 mg per kg of body weight, for example from 0.5 to 30 mg per kg of body weight, 1 to 20 mg per kg of body weight or 1 to 10 mg per kg of body weight.

An agent suitable for increasing life expectancy or treating a metabolic disease is one which lowers post-DEX cortisol concentration and increases pre- and post-DEX insulin and/or glucose levels and which increases total cholesterol levels. Preferably, the observed increase or decrease returns the level of the metabolite being monitored back to level comparable to the level observed in the absence of the ER22/23EK polymorphism.

The present invention also provides an agent identified by a method of the invention. An agent identified in the screening method of the invention may be used in the therapeutic or preventative treatment of a metabolic disorder or to increase life expectancy. The condition of a patient having a metabolic disorder can be improved by the administration of a therapeutically effective, non-toxic amount of such an agent. Accordingly, the present invention provides a method of treating a metabolic disorder or increasing life expectancy consisting essentially of administering a therapeutically inactive amount of an agent of the invention to a patient in need thereof.

An agent may be formulated with standard pharmaceutically acceptable carriers and/or excipients as it routine in the pharmaceutical art, and as fully described in Remington's Pharmaceutical Sciences, Mack Publishing Company, Eastern Pennyslavania 17^{th} Ed. 1985, the disclosure of which is included herein of its entirely by way of reference. A pharmaceutical composition comprising an agent of the invention and a pharmaceutically effective carrier or diluent is thus provided by the invention.

Compositions and medicaments for use in a method of treating a gastrointestinal disorder may be formulated in dosage form. Medicaments comprising a therapeutic agent identified by a method of the invention may be in a form suitable for administration to a patient, for example in table, capsule or liquid form, or may be in a concentrated form suitable for preparation by a pharmacist.

The formulation of the product for use in preventing or treating the disease will depend upon factors such as the nature of the agent identified and the disease to be prevented or treated. Typically the agent is formulated for use with a pharmaceutically acceptable carrier or diluent. For example it may be formulated for intracranial, parenteral, intravenous, intramuscular, subcutaneous, transdermal or oral administration. A physician will be able to determine the required route of administration for each particular patient. The pharmaceutical carrier or diluent may be, for example, an isotonic solution.

The dose of product may be determined according to various parameters, especially according to the substance used; the age, weight and condition of the patient to be treated; the route of administration; and the required regimen. A suitable dose may however be from 0.1 to 100 mg/kg body weight such as 1 to 40 mg/kg body weight. Again, a physician will be able to determine the required route of administration and dosage for any particular patient.

The following Examples illustrate the invention.

Subjects and Methods

Subjects

A total of 202 human subjects participated in the study. Their age varied between 53 and 82 years (98 men and 114 women with mean ages of 67.7±0.6 and 65.9±0.6 years, respectively). They were living in a suburb of Rotterdam, The Netherlands. These subjects were participants in the Rotterdam Study, a population-based cohort study (7983 subjects) of the determinants of chronic disabling diseases in the elderly and were selected at random. Subjects with acute, psychiatric or endocrine diseases, including diabetes mellitus treated with medication were not invited. Compared to all participants of the Rotterdam study, there were no differences in age and gender distribution and cardiovascular risk factors. The subjects gave their written consent to participate in the study, which received the approval of the Medical Ethics Committee of the Erasmus University Medical School. In order to get more information about the individual variability of the feedback sensitivity of the HPA-axis all 202 subjects were invited for a second DST with a lower dose DEX (0.25 mg) two and a half years later. 149 subjects agreed to participate in this second test (72 men and 77 women).

Anthropometric Measurements

Body weight, height and waist to hip ratio of the subjects were measured, and the body mass index (BMI, kg/m²) was calculated. Blood pressure was measured in sitting position at the right upper arm with a random-zero sphygmomanometer.

Dexamethasone Suppression Tests

The two dexamethasone suppression tests (DST) were performed as described previously (Huizenga et al, J.Clin.Endocrinol Metab.1998; 83(1): 47-54). In brief, venous blood was obtained in the morning between 8 and 9 am after an overnight fast for serum cortisol and insulin measurements. Participants were instructed to ingest a tablet of 1 mg (and 0.25 mg, respectively) DEX at 11.00 pm. The next morning fasting blood was drawn by venapuncture at the same time as the previous morning. To check for compliance and possible abnormalities in the metabolism of DEX, the DEX concentration was also measured in a radioimmunoassay using antiserum obtained from IgG Corporation (Nashville, Tenn.). Intra- and interassay variations were below 8.5% and 14.2% respectively.

Hormonal Measurements

Serum cortisol concentrations were determined using RIA-kits obtained from Diagnostics Products Corporation (Los Angeles, Calif.). Intra- and interassay variations were below 8.0% and 9.5% respectively. Circulating insulin and cortisol binding globulin concentrations were determined using commercially available ratioimmunoassays (Medgenix Diagnostics, Brussels, Belgium). Intra- and interassay variations were 8.0% and 13.7% respectively. Estradiol, androstenedione and DHEAS concentrations were determined using RIA-kits obtained from Diagnostics Products Corporation. Intra- and interassay variations; estradiol: 7.0% and 8.1% androstenedione: 8.3% and 9.2%, DHEAS: 5.3% and 7.0% SHBG was assayed with an commercially available immunoradiometric assay (Diagnostics Products Corporation; intra- and interassay variations were 3.6% and 6.9% respectively). Testosterone was measured with a non-commercial radioimmunoassay (intra- and interassay variations 3.6% and 6.9%). Commercially available immunoradiometric assays were used for the measurement of IGF-BP1 (Diagnostic System Laboratories Inc.; intra- and interassay variations 4.0% and 6.0%).

Biochemical Measurements

Glucose, total cholesterol, HDL-chlesterol and triglycerides were measured using standard laboratory methods. Low density lipoprotein (LDL)-cholesterol was calculated using the following formula: LDL-cholesterol=total cholesterol−((triglycerides/5)+HDL-cholesterol).

Genetic Analysis

Restriction fragment length polymorphism and analysis was carried out to determine GR genotypes. DNA was extracted from peripheral blood leukocytes using standard techniques. PCR amplification of the GR gene was carried out employing primer sequences and amplification conditions as described previously (Koper et al., Hum.Genet. 1997; 99(5): 663-8). The PCR-products were digested with 1 U Mnl I (New England Biolabs, Inc) at 37° C. for 1 hour. Mnl I cleaves at 5′-CCTC(N)7-′3 and at 3′-GGAG(N)6-′5. Fragments were visualised with ethidium bromide on a 3% agarose-gel (MP-Boehringer, Manneheim). We re-analysed the 18 heterozygous and 10 wild type samples and found identical genotypes.

Cardiovascular Tests

Assessment of carotid intima-media thickness and the presence of atherosclerotic plaques in the carotid artery was performed with ultrasonography of both carotid arteries with a 7.5-MHz linear-array transducer (ATL UltraMarkIV, Advanced Technology Laboratories, Bothwell, Wash.) as described previously (Bots et al., J Clin Epidemiol 1997;50(7):801-7 and Bots et al., Circulation 1997;96(5):1432-7). For each subject mean intima-media thickness (left+right/2) was taken as measure for wall thickness of the distal common carotid artery. The common and internal carotid artery and the carotid bifurcation were both on line and off line (from tapes) evaluated for the presence (yes/no) of atherosclerotic lesions on both the near and the far wall. Plaques were defined as a focal widening relative to adjacent segments, with protrusion into the lumen composed of either only calcified deposits or a combination of calcification and noncalcified material. A total carotid plaque score was defined by summation of the presence of plaques at the left and right side at three locations (score ranging from to 0 to 6) (Bots et al., J. Clin. Epidemiol 1997; 50(7):801 and Bots et al., Stroke 1997; 28 (12):2442-7). Measurement of the systolic blood pressure level of the posterior tibial artery at both the left and right side was performed to evaluate the presence of atherosclerosis in the arteries of the lower extremities (Meijer et al., Arterioscler Thromb Vasc Biol 1998; 352 (9128): 601-5). The ratio of the systolic blood pressure at the ankle to the systolic blood pressure at the arm was calculated for each leg. The lowest ankle-arm index in either leg was used in the analysis (Meijer et al., Arterioscler Thromb Vasc Biol 1998; 18(2): 185-92). Myocardial infarction was diagnosed on the basis of findings on a resting 12-lead electrocardiogram (ACTA Gnosis IV, EsaoteBiomedica) or self-report, which was verified by research physicians (Kors et al., Lancet 1998; 352; 9128:601-5 and de Bruyne et al., J Clin Epidemiol 1997; 50(8): 947-52). The presence of angina pectoris and intermittent claudication was assessed by a Dutch version of the Rose questionnaire (Rose et al., In: WHO: Geneva; 1982; 1982). Cardiovascular disease was defined as the presence of at least one of these three conditions.

Statistical Analysis

Data were analysed using SPSS for Windows, release 9.0 (SPSS, Chicago, Ill.). Logarithmic transformations were applied to normalize variables and to minimize the influence of outliers. Differences between the ER22/23EK-carriers and the non-carriers were adjusted for age and tested by NCOVA using the general linear model procedure. A paired samples t-test was used to compare changes in insulin and glucose concentrations before and after the administration of DEX in all subjects. Results are reported as mean±SE. Comparison of the frequencies of the genotypes between different age-groups was carried out using a Chi-square test. P values are two-sided throughout and a p<0.05 was considered to be significant.

Results

### EXAMPLE 1

### Identification of ER22/23EK Carriers

Restriction fragment length polymorphism analysis revealed in the study population of 202 subjects a total of 18 individuals (8.9%) who were heterozygous for the polymorphism in codon 22/23 (see also FIG. 2). No individuals homozygous for this polymorphism were found in this group. The allele frequency of the variant allele in this group was 4.2%. Genotype distributions did not differ from those expected under Hardy-Weinberg equilibrium conditions. Sexes were equally represented in the group of ER22/23EK-carriers, as well as in the group of non-carriers. The ER22/23EK-carriers were 2.7 years older compared to non-carriers, which did not reach statistical significance (Table 1; p-0.07). However, in the age group between 67 and 82 years the number of ER22/23EK-carriers was higher (12.9%) than in the age group between 53 and 67 (n=101, 4.9% ER22/23EK-carriers; p 0.05). To rule out the influences of differences in age, all parameters were adjusted for age. No significant differences in anthropometric parameters or blood pressure between the groups were present, as shown in Table 1. At the second examination after 2.5 years 149 of the initial 202 individuals participated (74%), 13 of whom were heterozygous for the codon 22/23 polymorphism. Also in this group of ER22/23EK-carriers the sexes were equally represented. The group of non-carriers now consisted of 66 men and 70 women.

TABLE 1
Anthropometric parameters and blood pressure in
non-carriers (n = 184) and ER22/23 EK-carriers (n = 18)
at baseline.

| | Non-carriers | | ER22/23EK-carriers | | |
|---|---|---|---|---|---|
| | Mean | SE | mean | SE | P-value* |
| Age (years) | 66.5 | 0.44 | 69.2 | 1.68 | 0.07 |
| Height (cm) | 1.70 | 0.01 | 1.69 | 0.02 | 0.85 |
| Weight (kg) | 74.7 | 1.15 | 71.9 | 1.97 | 0.69 |
| BMI (kg/m²) | 26.4 | 0.28 | 25.4 | 0.85 | 0.25 |
| WHR | 0.92 | 0.01 | 0.94 | 0.02 | 0.62 |
| SBP (mmHg) | 138.9 | 1.42 | 140.2 | 5.03 | 0.86 |
| DBP (mmHg) | 74.7 | 0.73 | 77.1 | 2.95 | 0.42 |

*Test for the difference between non-carriers and ER22/23EK-carriers. All parameters were log transformed and, with the exception of age, adjusted for age.
SE, Standard Error of the mean,
BMI, body mass index,
WHR, waist to hip ratio,
SBP, systolic bloodpressure,
DBP, diastolic bloodpressure.

### EXAMPLE 2

### Feedback Sensitivity of the HPA-Axis

Table 2a shows the concentrations of early morning serum cortisol concentrations before and administration of 1 mg DEX, the DEX concentration, and the cortisol suppression in reaction to DEX (Δ cortisol). There were no differences between the non-carriers and the ER22/23EK-carriers in fasting cortisol concentrations, nor in the decrease of serum cortisol concentrations after dexamethasone. However, the cortisol concentrations after the 1 mg DST were significantly higher in ER22/23EK-carriers than in non-carriers (57.9 in ER22/23EK-carriers and 33.3 nmol/l in non-carriers, p=0.01). The actual DEX concentrations did not differ in both groups, so the higher post DEX cortisol levels in the ER22/23EK-carriers were not due to differences in the metabolism of DEX. Also fasting cortisol-binding globulin (CBG) levels were not different in ER22/23EK-carriers and in non-carriers.

Table 2b provides the same parameters before and after the administration of 0.25 mg DEX. Again, there were no differences in fasting cortisol or decrease in cortisol. The cortisol concentrations after the administration of 0.25 mg DEX in ER22/23EK-carriers were not different from those in the non-carriers.

TABLE 2a
Cortisol and DEX concentrations in non-carriers (n = 184)
and in ER22/23EK-carriers (n = 18) before and after 1 mg
DEX at first examination.

| | Non-carriers | | ER22/23EK-carriers | | |
|---|---|---|---|---|---|
| | Mean | SE | mean | SE | P-value* |
| Fasting cortisol | 518.4 | 11.1 | 562.8 | 47.3 | 0.25 |
| (nmol/l) | | | | | |
| PostDEX cortisol | 33.3 | 4.9 | 57.9 | 17.5 | 0.01 |
| (nmol/l) | | | | | |
| Δ cortisol (nmol/l) | 481.4 | 11.5 | 504.9 | 48.3 | 0.79 |
| DEX (nmol/l) | 7.32 | 0.27 | 6.63 | 0.84 | 0.21 |

*Test for the difference between non-carriers and ER22/23EK-carriers. All parameters were log transformed and adjusted for age.
SE, Standard Error of the mean,
DEX, dexamethasone.

TABLE 2b
Cortisol and DEX concentrations in non-carriers (n = 136)
and in ER22/23EK-carriers (n = 13) before and after 0.25 mg
DEX at second examination.

| | Non-carriers | | ER22/23EK-carriers | | |
|---|---|---|---|---|---|
| | Mean | SE | mean | SE | P-value* |
| Fasting cortisol (nmol/l) | 545.3 | 12.4 | 527.2 | 30.3 | 0.82 |
| PostDEX cortisol (nmol/l) | 259.5 | 12.4 | 267.5 | 31.3 | 0.60 |
| Δ cortisol (nmol/l) | 285.8 | 13.5 | 259.7 | 40.7 | 0.26 |
| DEX (nmol/l) | 2.85 | 0.13 | 2.88 | 0.52 | 0.71 |

*Test for the difference between non-carriers and ER22/23EK-carriers. All parameters were log transformed and adjusted for age.
SE, Standard Error of the mean,
DEX, dexamethasone.

### EXAMPLE 3

### Insulin and Glucose Concentrations

Tables 3a and 3b show the fasting insulin and glucose concentrations before and after the administration of 1 and 0.25 mg DEX, respectively, and the change in insulin levels in response to DEX administration. In order to be certain that only the data from subjects with a normal carbohydrate tolerance were analysed, subjects developing either hyperinsulinaemia or diabetes mellitus after the inclusion in the study (fasting insulin or glucose concentrations above values of 25 mU/1 or 7.8 mmol/l, respectively) were excluded from this analysis (17 non-carriers excluded, n=167, and 3 ER22/23EK-carriers excluded, n=15). In all 182 subjects together, a significant increase in insulin concentrations in response to the administration of 1 mg DEX was noted (11.5±5.15 mU/L before, and 17.2±8.41 mU/L after DEX administration, respectively p<0.001). There were no differences in this increase in serum insulin concentrations between the control group and the ER22/23EK-carriers (5.7±0.6 versus 5.5±1.3 mU/L).

The fasting insulin concentrations tended to be lower in ER22/23EK-carriers than in non-carriers (p=0.06). The same applied to the fasting serum insulin levels measured after 1 mg DEX (p=0.07). These differences in post DEX insulin concentrations were not due to differences in DEX concentrations between the two groups. Fasting glucose concentrations were not different between the non-carriers and ER22/23EK-carriers (5.71±0.05 versus 5.69±0.16 mmol/L, respectively).

At second examination, 2.5 years later, the fasting insulin levels in ER22/23 EK-carriers were significantly lower than in the non carriers (p<0.001). Insulin levels decreased in the total group of 115 subjects after the administration of 0.25 mg DEX (14.7+0.45 before, and 13.9±0.50 mU/L after DEX administration, respectively, p=0.02). There were no differences in this decrease in insulin levels between the non-carriers and the ER22/23EK-carriers. After the administration of 0.25 mg DEX insulin concentrations were not different in the ER22/23EK-carriers from those in the non-carriers (p=0.11). Glucose concentrations decreased in all subjects in response to 0.25 mg DEX administration (5.6±0.06 mmol/l before, and 5.5±0.05 mmol/l after DST, respectively, p=0.004). Baseline glucose levels tended to be lower in the ER22/23EK-carriers than in the non-carriers (5.3±0.20 and 5.6±0.06 mmol/l, respectively; p=0.07). After the 0.25 mg DST there were no significant differences in glucose concentrations (ER22/23EK-carriers: 5.3±0.17 versus non-carriers; 5.5±0.05 mmol/l, respectively; p=0.11). There were no differences between the non-carriers and ER22/23 EK-carriers in change in glucose in response to DEX administration.

TABLE 3a
Insulin concentrations in non-carriers (n = 167) and
ER22/23EK-carriers (n = 15) before and after 1 mg DEX at
first examination

| | Non-carriers | | ER22/23EK-carriers | | |
|---|---|---|---|---|---|
| | Mean | SE | mean | SE | P-value* |
| Fasting insulin (mU/l) | 11.7 | 0.40 | 8.9 | 1.19 | 0.06 |
| Post DEX insulin (mU/l) | 17.4 | 0.67 | 14.4 | 1.74 | 0.07 |
| Δ insulin (mU/l) | 5.7 | 0.55 | 5.5 | 1.30 | 0.90 |
| Fasting glucose (mmol/l) | 5.71 | 0.05 | 5.69 | 0.16 | 0.68 |

TABLE 3b
Insulin and glucose concentrations in non-carriers (n = 105)
and ER22/23EK-carriers (n = 10) before and after 0.25 mg
DEX at second examination.

| | Non-carriers | | ER22/23EK-carriers | | |
|---|---|---|---|---|---|
| | Mean | SE | mean | SE | P-value* |
| Fasting insulin (mU/l) | 15.2 | 0.46 | 10.0 | 1.39 | <0.001 |
| Post DEX insulin (mU/l) | 14.2 | 0.52 | 10.0 | 1.28 | 0.11 |
| Δ insulin (mU/l) | −0.92 | 0.38 | 0.01 | 0.95 | 0.56 |
| Fasting glucose (mmol/l) | 5.60 | 0.06 | 5.30 | 0.20 | 0.07 |

*Test for the difference between non-carriers and ER22/23EK-carriers. All parameters were log transformed and adjusted for age.
SE, Standard Error of the mean. Subjects with fasting insulin >25 mU/l or fasting glucose >7.8 mmol/l were left out of the calculation; 3 ER22/23EK-carriers and 31 non-carriers were left out.

### EXAMPLE 4

### Risk Factors for Coronary Heart Disease and Diabetes Mellitus

In Table 4 serum concentrations of IGF-BP1, cholesterol, HDL-cholesterol and triglycerides are shown. There were no differences between non-carriers and ER22/23EK-carriers in IGF-BPI levels or in HDL-cholesterol and triglyceride concentrations. However, total cholesterol levels were significantly lower in ER22/23EK-carriers than in non-carriers (6.86 in non-carriers, versus 6.12 mmol/L in ER22/23EK-carriers p=0.02). At the second examination after 2.5 years serum cholesterol concentrations were again lower (6.61 in non-carriers, versus 5.64 mmol/L in ER22/23EK-carriers p=0.01, not shown in table).

TABLE 4
Risk factors for coronary heart disease and Diabetes Mellitus
at first examination in non-carriers (n = 184) and ER22/
23EK-carriers (n = 18)

| | Non-carriers | | ER22/23EK-carriers | | |
|---|---|---|---|---|---|
| | Mean | SE | mean | SE | P-value* |
| IGF-BP₁ (μg/L) | 19.3 | 1.59 | 18.8 | 2.91 | 0.57 |
| Cholesterol (mmol/L) | 6.86 | 0.09 | 6.12 | 0.25 | 0.02 |
| LDL-cholesterol (mmol/L) | 5.11 | 0.08 | 4.31 | 0.25 | 0.01 |
| HDL-cholesterol (mmol/L) | 1.36 | 0.03 | 1.43 | 0.14 | 0.63 |
| Triglycerides (mmol/L) | 1.91 | 0.07 | 1.93 | 0.33 | 0.67 |

*Test for the difference between non-carriers and ER22/23EK-carriers. All parameters were log transformed and adjusted for age.
SE, Standard Error of the mean.
IGF-BP1, insulin-like growth factor-binding protein-1,
LDL-cholesterol, low density lipoprotein-cholesterol
HDL-cholesterol, high density lipoprotein-cholesterol.

### EXAMPLE 5

### Sex Hormones

Table 5 shows the fasting concentrations of sex hormones for men and women separately. No differences between the non-carriers and the ER22/23EK-carriers in the concentrations of estradiol, SHBG, androstenedione, DHEA-S or testosterone were detected.

TABLE 5
Hormones at first examination in male (n = 84) and female
(n = 100) non-carriers and in male (n = 10) and female
(n = 8) ER22/23EK-carriers

| | Non-carriers | | ER22/23EK-carriers | | |
|---|---|---|---|---|---|
| | Mean | SE | mean | SE | P-value* |
| Men | | | | | |
| Estradiol (pmol/L) | 107.4 | 20.2 | 108.0 | 22.8 | 0.63 |
| SHBG (nmol/L) | 50.74 | 2.30 | 44.01 | 6.74 | 0.84 |
| Adion (nmol/L) | 6.60 | 0.34 | 6.26 | 0.69 | 0.94 |
| DHEA-S (μmol/L) | 4.01 | 0.27 | 3.80 | 0.46 | 0.76 |
| Testos (nmol/L) | 20.2 | 0.56 | 21.9 | 1.70 | 0.75 |
| Women | | | | | |
| Estradiol (pmol/L) | 82.38 | 5.26 | 79.67 | 16.26 | 0.96 |
| SHBG (nmol/L) | 57.55 | 3.18 | 76.92 | 16.07 | 0.21 |
| Adion (nmol/L) | 4.46 | 0.22 | 4.59 | 0.78 | 0.90 |
| DHEA-S (μmol/L) | 2.52 | 0.16 | 2.42 | 0.44 | 0.92 |
| Testos (nmol/L) | 1.37 | 0.06 | 1.92 | 0.64 | 0.49 |

*Test for the difference between non-carriers and ER22/23EK-carriers. All parameters were log transformed.
SE, Standard Error of the mean SHBG, sex hormone binding globulin,
Adion, androstenedione,
DHEAS, dehydroepiandrosterone-sulfate,
Testos, testosterone

### EXAMPLE 6

### Atherosclerosis and Cardiovascular Disease

Table 6 presents the prevalence of cardiovascular disease and atherosclerosis in non-carriers and ER22/23EK-carriers. A history of myocardial infarction or the presence of angina pectoris or intermittent claudication was defined as cardiovascular disease. In the control-group 33% had cardiovascular disease, while in the group of the ER22/23EK-carriers 20% were affected, but this was not statistically significant (p=0.20). When tested for each condition separately the differences where not significant either; intermittent claudication: non-carriers 2 (1.1%) vs ER22/23EK-carriers 0 (0%), p=0.90, angina pectoris: non-carriers 14 (8.0%) vs ER22/23EK-carriers 1 (5.9%), p=0.79, myocardial infarction: non-carriers 61 (34%) vs ER22/23EK-carriers 5 (28%), p=0.14). We found also no difference between the two groups in mean intima-media thickness of the carotid artery (0.76±0.01 mm in the non-carriers and 0.77+0.06 mm in the ER22/23EK-carriers; p=0.80) or mean ankle-arm index (1.1±0.02 in the non-carriers and 1.1±0.07 in the ER22/23EK-carriers; p=0.89). The genotype distributions of the prevalence of presence of plaques in the carotid artery is also shown in Table 6. The odds ratio for presence of plaques in the carotid artery in ER22/23EK-carriers was 0.42 (95% CI 0.2-1.1). After adjustment for age this odds ratio decreased and was statistically significant: 0.27 (95% CI 0.1-0.8). When we carried out an additional adjustment for cholesterol the odds ratio was 0.33 (95% CI 0.1-1.0).

TABLE 6
Parameters of atherosclerosis and cardiovascular disease in
non-carriers (n = 181) and ER22/23EK-carriers (n = 18)

| | |
|---|---|
| | ER22/ |
| Non-carriers | 23EK-carriers |

| | | |
|---|---|---|
| Cardiovascular disease* | 54 (33%) | 3 (20%) |
| Mean intima-media thickness | 0.76 ± 0.01 | 0.77 ± 0.06 |
| a. carotis(mm) | | |
| Mean ankle-arm index | 1.09 ± 0.02 | 1.09 ± 0.07 |
| Presence of plaques in the a. carotis | 119 (66%) | 8 (44%) |
| Odds Ratio | 1 (reference) | 0.27 (0.1-0.8) |
| Presence of plaques in the a. carotis | 1 (reference) | 0.33 (0.1-1.0) |

Values are means +/− standard errors or number of subjects (%). Relative risk is given with a 95% confidence interval.
^{a}reference group; subjects homozygous for 22/23ER allele (wild type), adjusted for age,
^{b}adjusted for age and cholesterol
*History of myocardial infarction or presence of angina pectoris or intermittent claudication. a. carotis, carotid artery

Discussion

In this population study in the elderly involving 202 individuals we found 18 subjects who were heterozygous for the ER22/23EK polymorphism (8.9%). The ER22/23EK-carriers had higher serum concentrations of cortisol after the administration of 1 mg DEX than non-carriers. Furthermore, ER22/23EK-carriers tended to have lower insulin levels before and after a 1 mg DST. These data were partially confirmed two and a half years later with a 0.25 mg DST. Fasting insulin concentrations were again lower in ER22/23EK-carriers and fasting glucose levels tended to be lower in ER22/23EK-carriers as well. These observations suggest that this polymorphism in the GR gene is associated with a slight resistance of the feedback regulation of the HPA-axis.

This relative resistance also results in a lower effect of cortisol on glucose metabolism, resulting in slightly lower glucose concentrations, as well as lower insulin levels. This rather beneficial metabolic profile is supported by the observation that total serum cholesterol concentrations were significantly lower in the ER22/23EK group than in the group of non-carriers. This was confirmed at the second examination. These outcomes of a relative GC resistance, together with the lower insulin, total cholesterol and slightly lower serum glucose concentrations, indicate that ER22/23EK-carriers have a healthier metabolic profile than non-carriers. Indeed, the relative risk of presence of atherosclerotic plaques in the carotid artery was significantly lower in ER22/23EK-carriers than in non-carriers when adjusted for age. After an additional adjustment for cholesterol this significance disappeared, which indicates that the lower cholesterol levels explain most of the lower risk on the presence of plaques in the carotid artery. In this respect, the fact that we found in the present study a significantly higher percentage of ER22/23EK-carriers in the older age group supports the finding of a beneficial metabolic effect of this GR polymorphism in codon 22 and 23.

We found no other differences between the genotypes in anthropometric parameters, blood pressure, and serum levels of IGF-BP1, HDL-cholesterol, triglycerides or sex hormones.

In summary, in this study we observed that subjects who were heterozygous for the 22/23EK allele had significantly higher post DEX cortisol concentrations, lower insulin and slightly lower glucose levels before the administration of DEX, as well as slightly lower post Dex insulin levels that subjects without this GR variant. Furthermore, ER22/23EK-carriers had lower total cholesterol levels and were overrepresented in the older age group. These data suggest that ER22/23EK-carriers are relatively more ‘cortisol-resistant’ than non-carriers, which results in a better metabolic health profile.

1. A method of determining the risk of an individual developing a metabolic disorder, the method comprising:
(i) detecting in a sample from the individual the presence or absence of the ER22/23EK polymorphism in the glucocorticoid receptor gene; and
(ii) determining the likelihood of the individual developing a metabolic disorder, wherein the presence of the ER22/23EK polymorphism is indicative of a low risk of developing the metabolic disorder and the absence of the ER22/23EK polymorphism is indicative of a high risk of developing the metabolic disorder.

2. A method according to claim 1 wherein the metabolic disorder is cardiovascular disease.

3. A method according to claim 1 wherein the metabolic disorder is glucose intolerance or diabetes mellitus.

4. A method of predicting the longevity of an individual, the method comprising:
(i) detecting in a sample from the individual the presence or absence of the ER22/23EK polymorphism in the glucocorticoid receptor gene; and
(ii) determining the life expectancy of the individual, wherein the presence of the ER22/23EK polymorphism is indicative of a long life expectancy.

5. A method of determining the dose of glucocorticoid for administration to an individual in need thereof, the method comprising:
(i) detecting in a sample from the individual the presence or absence of the ER22/23EK polymorphism in the glucocorticoid receptor gene; and
(ii) determining whether the dose of glucocorticoid for administration to the individual should be altered compared to the standard dosage, wherein the presence of the ER22/23EK polymorphism indicates that the dosage should be increased.

6. A method according to claim 5 wherein the individual is suffering from an allergic disease, an antoimmune diesease, an inflammatory disorder, a neoplastic disease, graft rejection, sarcoidosis, vitamin D intoxication, thyroid storm, septic shock, cerebral edema, altitude sickness, chronic bronchitis or emphysema.

7. A method according to claim 5 wherein the glucocorticoid is selected from Hydrocortisone, Prednisone, Prednisolone, Methylprednisolone, Triamcinolone, Dexamethasone, Budesonide, Betamethasone and Beclomethasone.

8. A method according to any one of the claim 1, 4 and 5 wherein step (i) comprises contacting a sample from the individual with a specific binding agent for the ER22/23EK polymorphism and determining whether the agent binds to the polymorphism.

9. A method according to claim 8 wherein the agent is a nucleotide binding agent.

10. A method according to claim 9 wherein the nucleotide binding agent is an oligonucleotide probe or primer.

11. A method according to claim 10 wherein the agent is a polypeptide binding agent.

12. A method according to claim 11 wherein the polypeptide binding agent is an antibody.

13. A method of determining whether a treatment regimen is suitable for an individual having a metabolic disorder, the method comprising:
(i) detecting in a sample from the individual the presence or absence of the ER22/23EK polymorphism in the glucocorticoid receptor gene; and
(ii) determining whether the treatment is suitable for the individual, wherein the suitability of the treatment depends on the presence or absence of the ER22/23EK polymorphism.

14. A method for diagnosing and treating an individual susceptible to a metabolic disorder, the method comprising:
(i) detecting in a sample from the individual the presence or absence of the ER22/23EK polymorphism in the glucocorticoid receptor gene; and
(ii) administering to an individual having the ER22/23KK polymorphism a therapeutically effective amount of an agent which prevents or treats the metabolic disorder.

15. A method for increasing the life expectancy of an individual, the method comprising:
(i) detecting in a sample from the individual the presence or absence of the ER22/23EK polymorphism in the glucocorticoid receptor gene; and
(ii) introducing into the individual an allele of the glucocorticoid receptor gene or a glucocortidicoid receptor, wherein said gene or polypeptide does not have said polymorphism.

16. A method for identifying an agent for use in the treatment of a metabolic disorder or for increasing life expectancy, the method comprising:
(i) contacting a glucocorticoid receptor polypeptide having the sequence shown in SEQ ID NO: 1 or a fragment thereof which includes the ER22/23EK polymorphism with a test agent;
(ii) monitoring binding of the test agent to the polypeptide; and
(iii) determining whether said test agent may increase life expectancy or be suitable for treating a metabolic disease, wherein for increasing life expectancy or treating a metabolic disease agent is one that binds to the polypeptide.

17. A method according to claim 16 wherien said glucocorticoid receptor polypeptide is in a non-human animal which is transgenic for a polynucleotide having the sequence shown in SEQ ID NO: 1.


SEQUENCE LISTING (4 sequences)

SEQ ID NO: 1 - DNA, 4788 bp, Homo sapiens
  CDS (133)..(2466)
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggaaaagggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 2 - protein, 777 aa, Homo sapiens [sequence omitted]

SEQ ID NO: 3 - DNA, 4788 bp, Homo sapiens
  CDS (133)..(2466)
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 4 - protein, 777 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK